(19) United States
(12) Patent Application Publication
(10) Pub. No.: US 2026/0306733 A1
(43) Pub. Date: Oct. 8, 2026

(54) A storage case for an electromechanical medicament delivery device

(71) Applicant: SHL Medical AG, Zug (CH)

(72) Inventors: Martin Michael Coyne, III, Boonton, NJ (US); Christopher James Franzese, Boonton, NJ (US); Alejandra Linares Martinez, Boonton, NJ (US); James Thomas Hawthorne, Boonton, NJ (US); Madeleine Hayden Anderson, Boonton, NJ (US); Demosthenis Katsaros, Boonton, NJ (US); Amy Catherine Rinaldi, Boonton, NJ (US); Nils Weber, Zug (CH)

(21) Appl. No.: 19/473,833

(22) PCT Filed: Mar. 27, 2024
(86) PCT No.: PCT/EP2024/058328

§ 371 Date: Oct. 9, 2025

(30) Foreign Application Priority Data
Apr. 11, 2023 (EP) 23386026.1

(51) Int. Cl. A61M 5/00; A61M 5/315; H02J 7/82; H02J 7/96

(52) U.S. Cl. CPC A61M 5/002; A61M 5/31576; H02J 7/82; H02J 7/96; A61M 2005/31588; A61M 2205/3576; A61M 2205/3606; A61M 2205/60; A61M 2205/8206; A61M 2205/8237; A61M 2209/06

(57) ABSTRACT

The present disclosure generally relates to medicament delivery devices such as autoinjectors or large volume injection devices, and particularly concerns storage case for an electromechanical medicament delivery device.

### TECHNICAL FIELD

The present disclosure generally relates to medicament delivery devices such as autoinjectors or large volume injection devices, and particularly concerns storage case for an electromechanical medicament delivery device.

### BACKGROUND

A number of medical conditions require injections. Today, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning storage and electrical charging of electromechanical medicament delivery devices, which are set out in more detail below.

### SUMMARY

An object of the present disclosure is to provide a storage case for a medicament delivery device which solves, or at least mitigates problems of the prior art.

According to a first aspect of the present disclosure, there is provided a storage case for an electromechanical medicament delivery device, the storage case comprising: an outer protective housing; a device storage portion for an electrically rechargeable electromechanical medicament delivery device, an internal battery and a charging circuitry for charging of the internal battery of the storage case and a battery of the medicament delivery device when housed in the device storage portion, a charging input device connected with the charging circuitry of the internal battery and with a charging interface in the device storage portion; communication equipment configured to communicate with the medicament delivery device, and controller circuitry configured to: determine the state of charge of the internal battery; determine that the medicament delivery device requires charging, in case the state of charge of the internal battery exceeds a threshold, control the charging circuitry to charge a battery of the medicament delivery device, control at least one indicator of the medicament delivery device to indicate the charge status of the medicament delivery device, the indicator being configured to indicate injection status when the medicament delivery device is out of the storage case.

Embodiments of the present disclosure advantageously provides for improvements in charging and monitoring the charge level of reusable battery driven medical delivery devices. The storage case allows portability of the medicament delivery device while at the same time ensuring charging in a desired location. Furthermore, a context-based indication is provided where the same indicators are used for indicating the charging status and the injection progress or status during injection. This provides for reduced device complexity and cost while at the same time not compromising the usability of the medicament delivery device.

The charging circuitry can be a contact charging circuit, a contactless charging circuit or a combination thereof. In one embodiment where the charging circuitry comprises a contact charging circuit, the storage case comprises a contact port to be in contact with a corresponding electromechanical contact port of the electromechanical medicament delivery device. Alternatively, when the charging circuitry comprises a contactless charging circuit, e.g., an RF charging circuit, the electromechanical medicament delivery device doesn't need to comprise a corresponding charging port and the electromechanical medicament delivery device doesn't need to be placed in the storage case in a certain orientation. As a result, the storage case can be compatible with different types of electromechanical medicament delivery devices.

An electromechanical medicament delivery device typically has a reusable drive unit with motor, battery, and supporting circuits, intended for use with a disposable drug unit containing one or more doses of medication. The storage case is adapted for storing the reusable component in between medication administration intervals.

The state of charge is a measurable quantity that indicates the charge level of the battery. State of charge is typically expressed in a percentage of the full battery capacity and is measurable by for example analysing an open circuit voltage across the poles of the battery.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 301 relative to an axis 102, typically a central axis extending in the direction of the longest extension of the device and/or component. Similarly, “radial” or “radially” refer to a direction 302 extending radially relative to the axis, and “rotation”, “rotational” and “rotationally” refer to rotation relative to the axis.

According to one embodiment, the controller circuitry may be further configured to: detect that no medicament delivery device is in the device storage portion; determine that the state of charge of the internal battery is below a recharging limit, control the charging circuit to recharge the internal battery with power provided at the charging input device, and control at least one of the indicators to indicate the charge status of the internal battery only if the medicament delivery device is not simultaneously used for an injection event. In other words, the controller circuitry may cause the charging circuitry to replenish charge in the storage case internal battery so the medicament delivery device may be recharged once replaced in the case. The controller circuitry, based on interrogation of the charging case battery level, may optionally adjust the charging rate or profile, which may be advantageous to extend battery life. As the process proceeds, visual feedback as to the charge status, or the completion of charging, may optionally be provided to a user of the apparatus if the medicament delivery device indicator is presently not used for an injection event.

According to one embodiment, the controller circuitry may control the charging circuit to recharge the battery of the medicament delivery device only when the medicament delivery device is not simultaneously used for an injection event. This ensures that the indication of charging status and injection status are context based. Thus, charging is only performed and indicated in case injection is not in progress.

According to one embodiment, the controller circuitry may be further configured to: if/when the medicament delivery device is in the device storage portion, and the charging input device receives electrical power from an external power source, control a switching circuitry to bypass the internal battery and provide charging power directly to the medicament delivery device. That is, the battery of the medicament delivery device is prioritized to ensure that sufficient power for an injection is available in the battery of the medicament delivery device.

According to one embodiment, the controller circuitry being further configured to: determine that an injection event is due within a predetermined time, and control the charging circuitry to charge the battery of the medicament delivery device to at least a minimum state of charge required for the next injection event. Advantageously, an injection event is anticipated, for example based on data indicating an injection or administering plan for the specific drug at hand, the data may be accessible by reading a tag of the drug container. By anticipating the injection event, the charging may be planned thereafter.

According to one embodiment, the storage case may comprise a global positioning system. This allows location tracking capabilities for the device, case, or both. This is useful to avoid misplacement of the medicament delivery device and case, when the storage case is used for travel, or when patients want to verify they have the device and case before they return from travel. The GPS may also be useful in certain disease states characterized by cognitive decline, such as Alzheimer's disease. Furthermore, sound emission, haptic feedback, and visual location on a digital map may be implemented for localization. The digital map may be accessed through a drug delivery device app, through a website or app that works specifically with the storage case.

According to one embodiment, the storage case may comprise a wireless communication device configured to receive and transmit data to a remote server, the controller circuitry may be further configured to: collect injection data from the medicament delivery device, and transmit the injection data using the wireless communication device to the remote server. Thereby, injection data may be accessible to a medical expert through the cloud.

According to one embodiment, the controller circuitry may be further configured to: receive firmware updates for the medicament delivery device using the wireless communication device, update the firmware of the medicament delivery device. Accordingly, the medicament delivery device may remotely receive software updates that improve patient safety and also for pharmaceutical compliance purposes.

According to one embodiment, the controller circuitry may be further configured to compare the current installed firmware upon the medicament delivery device to the received firmware update. This allows for determining whether an update is needed or to determine specific portions of the firmware that needs to be, or that can be updated.

According to one embodiment, the storage case may comprise a drug storage portion for storage of a medicament container inside the protective housing. Thus, a combined storage case for both the medicament and the medicament delivery device is provided.

According to one embodiment, the storage case may comprise a refrigerator device to refrigerate the drug storage portion. This allows storage of medal drugs under suitable conditions.

It is understood that the electromechanical drug delivery device may be configured to deliver medicament to a patient irrespective of whether the storage case is used. That is, even if the storage case is not used a drug delivery can be provided using the electromechanical drug delivery device. The electromechanical drug delivery device can work independent of the storage case.

According to one embodiment, the storage case may comprise two portions reversibly separable from each other by a connection mechanism, wherein a first portion comprises the device storage portion and a second portion comprises a drug storage portion. This allows for separate storage of the drug storage portion in e.g., a fridge without having to place also the medicament delivery device in the same environment.

According to one embodiment, the first portion may further comprise a power plug for directly connecting to the electrical mains for charging of the medicament delivery device. The power plug may be an integral main connection that allows for direct connection to the mains.

According to one embodiment, the storage case may comprise a retractable power cord between the power plug and the first portion, wherein the power plug is releasable attached to the first portion. The cord allows for connecting the charging circuitry to the mains even if the storage case is some distance from the mains connection. Furthermore, the retraction of the cord facilitates storage of the cord out of the way when it is not needed.

According to one embodiment, the protective housing may comprise an at least partly light transmissive window through which the at least one indicator on the medicament delivery device is visible from outside the protective housing. Advantageously, the charge status of the medicament delivery device may then be monitored without opening the storage case.

According to one embodiment, the storage case may comprise a storage unit comprising the device storage portion and a drug storage portion, the storage unit is slidable into the protective housing, thereby allowing for storage of the drug delivery device and the medicament container in the same storage unit that is smoothly slidable in a common protective housing.

According to one embodiment, the storage case may comprise a solar panel configured to covert light to electrical energy to electrically charge the internal battery or the medicament delivery device.

According to one embodiment, the storage case may comprise an ultra-violet sterilizing device exposing the medicament delivery device to ultra-violet light during device storage. This allows for sterilizing the device between use.

According to one embodiment, the storage case may comprise: at least one storage compartment for drug cassettes and RFID antennas, the RFID antennas are configured to communicate with RFID chips on the drug cassettes to communicate information to the medicament delivery device. The information is related to the medicament delivery device and may include e.g., medication, dose, dosing frequency, number of cassettes per dosing interval, expiry date or lot information.

According to a second aspect of the present disclosure, there is provided a method for controlling charging of an electrically rechargeable medicament delivery device storable in a storage case comprising: a device storage portion for accommodating the electrically rechargeable electromechanical medicament delivery device, and an internal battery and charging circuitry for charging of the medicament delivery device when housed in the device storage portion, the method comprising: determining the state of charge of the internal battery; determining that the medicament delivery device requires charging, in case the state of charge of the internal battery exceeds a threshold, charging a battery of the medicament delivery device, and controlling at least one indicator of the medicament delivery device to indicate the charge status of the medicament delivery device, the indicator being further configured to indicate injection status when the medicament delivery device is out of the storage case.

According to one embodiment, the method may further comprise: detecting that no medicament delivery device is in the device storage portion; determining that the state of charge of the internal battery is below a recharging limit, charging the internal battery with power provided at a charging input device connected to external mains power, and controlling at least one of the indicators to indicate the charge status only if the medicament delivery device is not simultaneously used for an injection event.

To charge from external mains includes any intermediate devices and adapters. For example, a USB wall hub may be plugged into mains and then connected to the charging port at lower voltage than directly from mains. For instance, 240 VAC at USB hub, then 5 VDC to charging case via USBC. Thus, the charge port of the storage case may be connected to the mains via intermediate transformers and/or adapters, and/or chargers.

According to one embodiment, the method may further comprise: if the medicament delivery device is in the device storage portion, and if electrical power is being received from an external power source, controlling a switching circuitry to bypass the internal battery and provide charging power directly to the medicament delivery device.

According to one embodiment, the method may further comprise: determining that an injection event is due within a predetermined time, and charging the battery of the medicament delivery device to at least a minimum state of charge required for the next injection event.

According to one embodiment, the method may further comprise: receiving a request for a present location of the storage case from a secondary electronic device; determining the present location using a global positioning system of the storage case, and transmitting, from the storage case, a digital message to the secondary electronic device including information indicating the present location.

According to one embodiment, the method may further comprise: charging the battery of the medicament delivery device only when the medicament delivery device is not simultaneously used for an injection event.

Further effects and features of the second aspect of the present disclosure are largely analogous to those described above in connection with the first aspect of the present disclosure.

According to a third aspect of the present disclosure, there is provided processing circuitry configured to control charging of an electrically rechargeable medicament delivery device storable in a storage case comprising: a device storage portion for accommodating the electrically rechargeable electromechanical medicament delivery device, and an internal battery and charging circuitry for charging of the medicament delivery device when housed in the device storage portion, the processing circuitry is configured to: determine the state of charge of the internal battery; determine that the medicament delivery device requires charging, in case the state of charge of the internal battery exceeds a threshold, charging a battery of the medicament delivery device, and control at least one indicator of the medicament delivery device to indicate the charge status of the medicament delivery device, the indicator being further configured to indicate injection status when the medicament delivery device is out of the storage case.

Processing circuitry may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device. The processing circuitry may have access to a non-transitory computer-readable storage medium storing instructions that when executed by the processing circuitry causes the execution of the method described herein.

Further effects and features of the third aspect of the present disclosure are largely analogous to those described above in connection with the first aspect and the second aspect of the present disclosure.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an electromechanical autoinjector according to embodiments of the present disclosure;

FIG. 2 is a block diagram of a storage case according to embodiments of the present disclosure;

FIG. 3A-B is a flow-chart of method steps according to embodiments of the present disclosure;

FIG. 4 is a block diagram of a storage case according to embodiments of the present disclosure;

FIG. 5A is a perspective view of a storage case according to embodiments of the present disclosure;

FIG. 5B is a perspective view of a storage case according to embodiments of the present disclosure;

FIG. 6A is a perspective view of a storage case according to embodiments of the present disclosure;

FIG. 6B is a perspective view of a storage case according to embodiments of the present disclosure;

FIG. 7A is a perspective view of a storage case according to embodiments of the present disclosure;

FIG. 7B is a perspective view of a storage case according to embodiments of the present disclosure;

FIG. 7C is a perspective view of a storage case according to embodiments of the present disclosure;

FIG. 8A are perspective views of a storage case according to embodiments of the present disclosure;

FIG. 8B are perspective views of a storage case according to embodiments of the present disclosure;

FIG. 8C are perspective views of a second portion of a storage case according to embodiments of the present disclosure;

FIG. 8D is a perspective view of a first portion of a storage case according to embodiments of the present disclosure;

FIG. 8E is a perspective view of a first portion of a storage case according to embodiments of the present disclosure;

FIG. 9A are perspective views of a storage case according to embodiments of the present disclosure;

FIG. 9B are perspective views of a storage case according to embodiments of the present disclosure;

FIG. 10 are perspective views of a storage case according to embodiments of the present disclosure;

FIG. 11 is a perspective view of a storage case according to embodiments of the present disclosure;

FIG. 12A is a cross-section of a storage case according to embodiments of the present disclosure;

FIG. 12B are perspective views of the storage case in FIG. 2A;

FIG. 12C are perspective views of a storage case according to embodiments of the present disclosure;

FIG. 13A are perspective views of a storage case according to embodiments of the present disclosure;

FIG. 13B is a perspective view of a device storage portion according to embodiments of the present disclosure, and

FIG. 14 is a flow chart of method steps according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

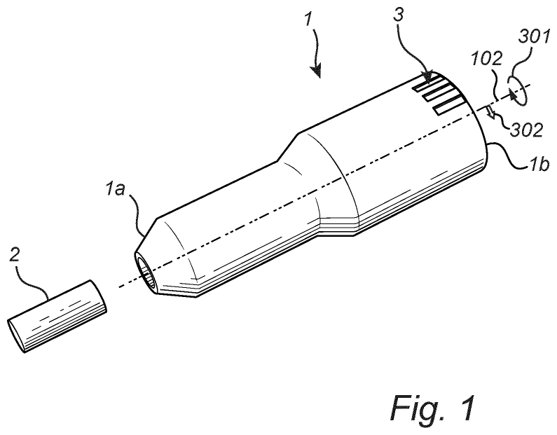

FIG. 1 shows an example of an electromechanical medicament delivery device 1 such as an autoinjector according to embodiments of the present disclosure. The medicament delivery device 1 is configured to expel medicament from a medicament container 2 via a medicament delivery member such as a needle, to a user at a dose delivery site. The medicament delivery device 1 extends from a proximal end 1a to a distal end 1b relative to the axis 102.

The medicament delivery device 1 is a reusable drive unit with motor, battery, and supporting circuits, intended for use with the disposable container 2 containing one or more doses of medication. Patients may store the reusable device 1 in between medication administration intervals. The medicament delivery device 1 comprises indicators 3 to provide users feedback on e.g., the device readiness, battery charge status, injection process, and injection.

However, the inventors identified several challenges with reusable medication delivery devices. For example, reusable medication delivery devices powered by rechargeable batteries require periodic recharging.

Furthermore, confusion may arise if the same visual indicator is used for dose delivery progress and charging status. If different indicators are used, usability may improve, but medication delivery device complexity (and cost) increases, as more indicators are needed in/on the device. Thus, improved solutions are needed to allow a user to unambiguously discern injection and charge progression, particularly based on the use context of the medication delivery device: charging, injection, or some other state.

In addition, medication delivery device charging may take place in a different location from medication administration. For instance, patients may also want to charge the medication delivery device in one location (e.g., kitchen or bedroom) and administer in a more private location (e.g., the bathroom).

Appropriate electrical plugs may not be available in the injection location, or there may be insufficient storage space or cord length for the medication delivery device while charging (e.g., bathroom vanity or counter); patients may also be concerned about getting the device wet, or damage to the device during charging. Patients may also wish to travel with the medication delivery device to a location where electrical power is not readily available. Thus, improved solutions are needed to ensure the delivery device may be charged in a desired location while allowing portability and discretion during administration, anticipating the charging and administration locations may be different.

Reusable medication delivery devices are stored between injection intervals. These intervals may often be lengthy, especially as dosing intervals stretch to once-monthly, once every two months, once every three months, once every six months, or even longer. This can result in misplacement of the reusable medication delivery device, loss of charge, or exposure to dirt, dust, or fluids between injections that may cause malfunction or improper operation. Thus, there is a need for improved solutions to store the medication delivery device, to ensure it is charged for each medication dosing interval, and to charge the device while allowing portability and discretion.

At least some of the above challenges are addressed by a storage case which will now be described in more detail with reference to subsequent drawings.

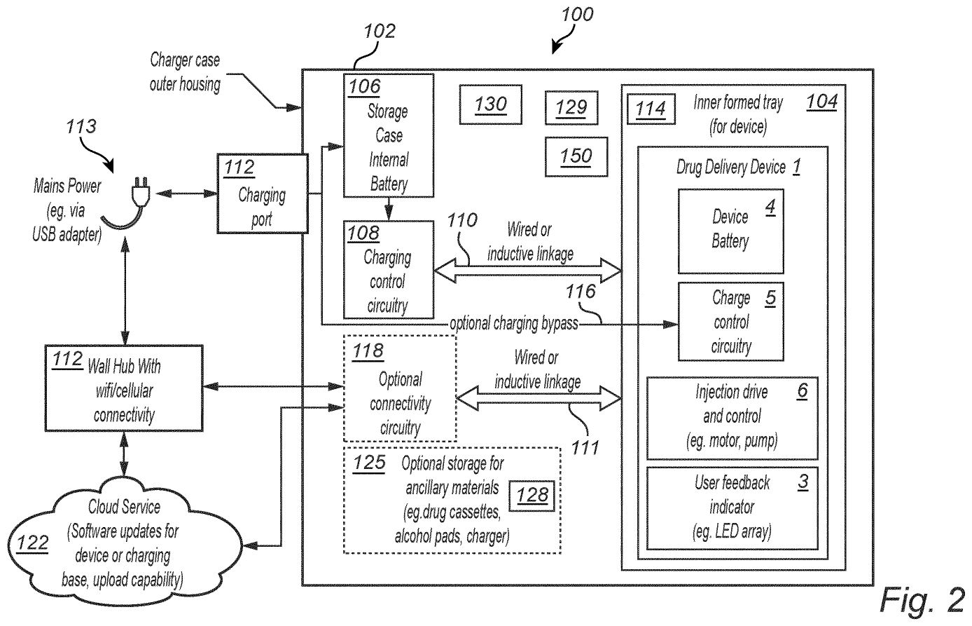

FIG. 2 is a block diagram of a storage case 100 for an electromechanical medicament delivery device 1. The storage case 100 comprises an outer protective housing 102 and a device storage portion 104 for accommodating the electrically rechargeable electromechanical medicament delivery device 1. In some embodiments, the device storage portion 104 comprises an inner formed tray in which the electromechanical medicament delivery device 1 snuggly fits.

The electromechanical medicament delivery device 1 comprises a battery 4 and a charging control circuit 5 that receives power from a source external to the electromechanical medicament delivery device 1 and control charging of the battery 4.

The electromechanical medicament delivery device 1 further comprises user feedback indicators 3 such as an array of light emitting diodes.

Additionally, the electromechanical medicament delivery device 1 comprises suitable injection drive and control devices 6 including motors and pumps for driving an injection event. Further, the medicament delivery device 1 may comprises a medication container or medication container interface, and optionally, an integral delivery needle. A new medication container is used at each medication administration interval with the reusable.

The storage case 100 comprises an internal battery 106 and a charging circuitry 108. The charging circuitry 108 is configured to charge the internal battery of the storage case and in some cases the battery 4 of the medicament delivery device 1 when the medicament delivery device 1 is housed in the device storage portion 104. Charging of the battery 4 of the medicament delivery device 1 may be performed via a wired connection of an inductive linkage, generally indicated as 110.

The storage case 100 further comprises a charging input device 112 connected with the charging circuitry 108 of the internal battery and with a charging interface 114 in the device storage portion 104. The charging interface 114 may be provided as electrical leads connectable with mating leads of the medicament delivery device 1. The charging input device 112, e.g., a charging port is connectable to the mains 113.

The charging circuitry 108 is configured to regulate voltage and charge transfer from the internal battery 106 of the storage case to the battery 5 of the medicament delivery device. The charging circuitry 108 may further provide voltage regulation of incoming power to the storage case to an acceptable level to enable recharging of the internal storage case battery 106 and/or charging of the stored medication delivery device 1 through direct electrical contact or inductive charging. Charging control circuitry 108 may also include overcurrent protection as well as charge rate control (e.g., “fast” or “burst” charging, or slow “trickle” charging, or combinations thereof) for either the storage case battery or device battery, as well as one or more input/output devices to allow a user to monitor and/or control aspects of the charging and/or charge state.

The charging circuitry 108 may also be provided with optional bypass capability 116, wired or inductive, allowing the case to preferentially recharge the device battery 4 if it is in a depleted state (e.g., as detected by one or more of the charge control circuits within the apparatus). The charging circuitry 108 may also be provided in the charging case for bidirectional connectivity to an outside data source (e.g., plug-in wall hub or direct-to-cloud service) to provide firmware updates to the case (and/or device therein) or serve as a data upload hub to move data from the device to the cloud after the device is used for injection.

The storage case 100 further comprises an optional communication arrangement 118 that is configured to transfer data from an external hub 120 or cloud-based services 122. The communication arrangement 118

Is provided in the storage case 100 for bidirectional connectivity to an outside data source, e.g., plug-in wall hub 120 or the direct-to-cloud service 122, to provide firmware updates to the storage case 100, and/or to the medicament delivery device 1, or serve as a data upload hub to move data from the medicament delivery device 1 to the cloud 122 after the medicament delivery device 1 is used for injection.

The storage case 100 may optionally comprise a drug storage portion 125 for storage of a medicament container inside the protective housing. The storage portion 125 may equally well adapted used for storage of e.g., a charger, pads, or other ancillary materials.

The drug storage portion 125 may be equipped with a refrigerator device 128 to refrigerate the drug storage portion 125 to allow for maintaining the medicament at a suitable temperature.

The storage case optionally comprises a global positioning system 129.

The storage case 100 further comprises a controller circuitry 130 configured to control charging of the internal battery 106, the battery 4 of the device 1, and other functionalities and components of the storage case 100.

The controller circuitry 130 is configured to determine the state of charge of the internal battery 106. Determine that the battery 4 of the medicament delivery device 1 requires charging. In case the state of charge of the internal battery 106 exceeds a threshold, control the charging circuitry 108 to charge a battery 4 of the medicament delivery device. Control at least one indicator 3 of the medicament delivery device 1 to indicate the charge status of the medicament delivery device battery 4, the indicator being configured to indicate injection status when the medicament delivery device is out of the storage case. It should be noted that in one example, the medicament delivery device can be automatically charged by the charging circuitry when the electric potential of the internal battery is higher than the electric potential of the battery of the medicament delivery device. Alternatively or additionally, the controller circuitry is configured to actively control the charging circuitry to charge the battery of the medicament delivery device only when the electric potential of the internal battery 106 exceeds a predetermined threshold; this example can make sure the internal battery is able to provide the electricity to the storage case for predetermined functions. Thus, the predetermined threshold can be designed based on how many and what functions the storage case should provide during charging the medicament delivery device.

Further, in some embodiments, the controller circuitry 130 is configured to collect injection data from the medicament delivery device 1. The controller circuitry is configured to use the communication device 118 to transmit the injection data using the remote server 122 or to the wall hub 120.

The controller circuitry 130 may further be configured to receive firmware updates for the medicament delivery device using the wireless communication device, and update the firmware of the medicament delivery device. Optionally, the controller circuitry 130 compares received firmware with the present firmware of the medicament delivery device prior to updating the firmware.

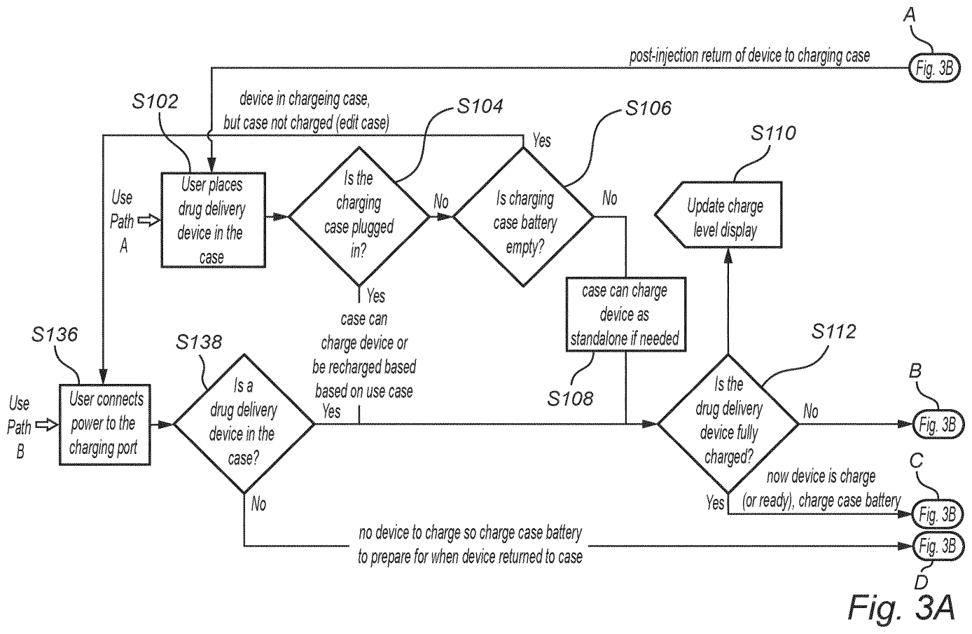

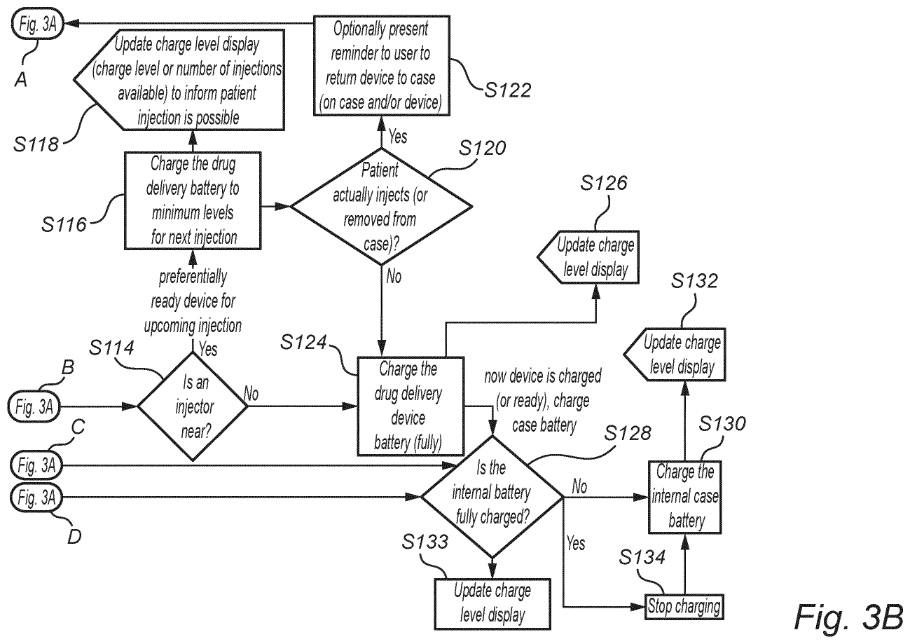

Turning now to the flow-chart of FIGS. 3A and 3B steps to charge a drug delivery device using the storage case under a variety of common scenarios that may occur in use. The links between the flow-charts are as follows, the reference A in FIG. 3A is connected with corresponding reference A in FIG. 3B, the reference B in FIG. 3A is connected with corresponding reference B in FIG. 3B, the reference C in FIG. 3A is connected with corresponding reference C in FIG. 3B, and the reference D in FIG. 3A is connected with corresponding reference D in FIG. 3B.

Starting from use path A, where the user has placed, S102, the medicament delivery device in the storage case. Placing the reusable medication delivery device 1 in the storage case 100 creates electrical contact with the internal battery 106 and charging control circuitry 108, which allows interrogation of the state of the medicament delivery device 1 and to recharge the batteries 4 within the medicament delivery device 1. Electrical contact may take place through conductive elements (e.g., pogo pins or wipers) or through inductive coupling.

The controller circuitry 130 determines whether the charge port 112 of the storage case 100 is connected to the mains 113 in step S104. In case the charge port 112 is not connected to the mains, the charge status of the internal battery 106 of the storage case 106 is evaluated in step S106. If the state of charge of the internal battery 106 is sufficiently high, the control circuitry control charging of the battery 4 of the device 1 using power from the internal battery 106 in step S108.

In step S110, the charge status, is indicated using the indicators 3 of the device 1.

The charge level is monitored in step S112, and different context-based charge actions are taken based on upcoming events. If case the battery 4 of the device 1 is not fully charged, the method proceeds to step S114 in FIG. 3B, and where the control circuitry 130 determines that an injection event is due within a predetermined time. In response, the control circuitry 130 controls the charging circuitry 108 to charge the battery 4 of the medicament delivery device 1 to at least a minimum state of charge required for the next injection event in step S116. The indicators 3 are controlled to indicate, S118, the new charge status, if the are not presently used for indicating injection status such as number of coming injections, or injection progress.

With the battery 4 at or above the minimum state of charge, it may be detected by the control circuitry 130, that patient removes the device 1 from the storage case 100, for example the connection to the charging circuitry 108 is lost, in step S120. It may be assumed that the device 1 was removed for performing an injection event. As an optional step, the device 1 or the storage case 100 may provide a reminder via indicators such a LEDs, or haptic, or audio, to the user to return the device 1 to the storage case 100 in step S122. Form here, the flow-chart returns to step S102 in FIG. 3A.

If the medicament delivery device 1 is not removed from the storage case 100, charging of the battery 4 of the medicament delivery device 1 is continued until fully charged, S124, and the indicators 3 are updated accordingly, in step S126.

Once the battery 4 of the medicament delivery device 1 is fully charged, or if the injection event in step S114 is not near, the charge state (state of charge) of the internal battery 106 of the storage case 100 is determined in step S128.

If the internal battery 106 is not fully charged, and the charging port 112 is connected to the mains 113, charging of the internal battery 106 is initiated in step S130. The charging status is indicated, S132, S133, and charging is stopped, S134, when the internal battery 106 is full, or when the mains 113 are disconnected from the charging port 112.

If, in step S112 in FIG. 3A, it is determined that the battery 4 of the medicament delivery device is fully charged, the method proceeds via link C to step S128 in FIG. 3B that was discussed above.

Now considering use path B in FIG. 3A. Here, the user connects the mains 113 to the charging port 113 in step S136. If the medicament delivery device 1 is in the storage case 100 connected to the charging circuitry 108 in step S138, the method proceeds to step S112 as was discussed above and the proceeding steps will not be repeated here.

If no medicament delivery device is in the device storage portion in step S138, the method proceeds to step S128, also discussed in more detail above.

For example, the controller circuitry 130 may be configured to detect, in step S138, that no medicament delivery device 1 is in the device storage portion 104. The controller circuitry may proceed to determine that the state of charge of the internal battery 106 is below a recharging limit in step S128. In response, the controller circuitry 130 control the charging circuit 108 to recharge the internal battery 106 with power provided at the charging input device 112. The controller circuitry 130 controls at least one of the indicators 3 to indicate the charge status of the internal battery 106, only if the medicament delivery device is not simultaneously used for an injection event.

Preferably, the controller circuitry 130 control the charging circuit 108 to recharge the battery 4 of the medicament delivery device 1 only when the medicament delivery device 1 is not simultaneously used for an injection event.

Returning to the flow chart in FIG. 3A. If the controller circuitry 130 determines that the medicament delivery device 1 is in the device storage portion 104 and the charging input device 112 receives electrical power from an external power source 113, step S136, S138 of use path B, the control circuitry may control a switching circuitry to bypass the internal battery 4 and provide charging power to the medicament delivery device 1 for performing the charging in steps S124 and/or S116. This is particularly useful the device battery 4 is in a depleted state or near depleted state.

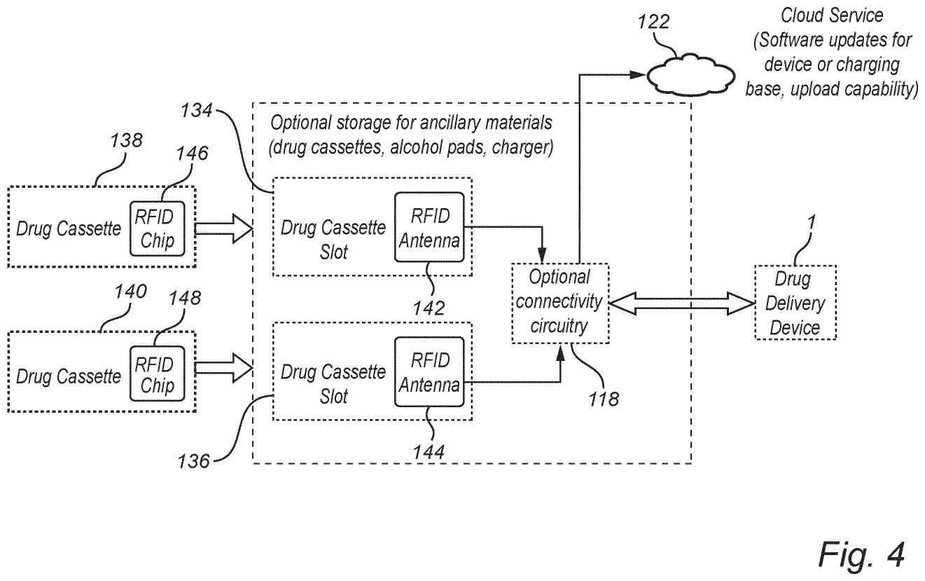

FIG. 4 illustrates a portion of the optional storage portion 125 shown in FIG. 2. The storage portion 125 comprises at least one storage compartment 134, 136 for drug cassettes 138, 140 and RFID antennas/readers 142, 144. The RFID antennas 142, 144 are configured to communicate with (i.e., read information from, and/or write information to) RFID chips 146, 148 on the drug cassettes 138, 140 to communicate information such as e.g., medication, dose, medication volume, medication concentration, dosing frequency, number of cassettes per dosing interval, an aspect of a multi-drug injection regimen, ordinal medication sequence, number of medications within a sequence, expiry or lot information, medication dose delivered, error states during medication delivery, etc to the medicament delivery device 1. The information may be processed by the connectivity software of the connectively circuitry 118, and optionally communicated (or verified) against the cloud database 122 and/or drug delivery device 1. The storage compartments may be slots or spaces for one or more drug cassettes. The drug cassettes 128, 140 and a cooperating feature in the optional storage compartments 134, 136 may be keyed or otherwise configured to orient the RFID chip 146, 148 over the antenna 142, 144 in a predictable manner for reading by the storage case 100. The controller circuitry 130 of the storage case 100 can also be configured to detect absence of one or more cassettes, either for a dosing interval requiring multiple cassettes where only one is present, or where no cassettes are present within an upcoming dosing interval.

It is further envisaged that the drug delivery device 1 may be equipped with an RFID chip, or equally RFID tag, may also be on the drive unit. The RFID chip on the drug delivery device 1 may be configured for identification and/or authentication of the drug delivery device 1 by the storage case 100.

Moreover, the storage case 100 may also comprise an RFID chip that can be read by an RFID reader of the drug delivery device 1. The RFID chip of the storage case may be configured for e.g. for identification/authentication of the storage case 100 by drug delivery device 1.

It is envisaged that the information on the RFID chips 146, 148 of one or multiple drug cassettes 138, 140 in the storage case may be overwritten by the RFID reader/writer of the storage case 100 e.g., modifying the used/unused information, injection parameters (injection speed, etc) or expiry date stored on the RFID chips 146, 148.

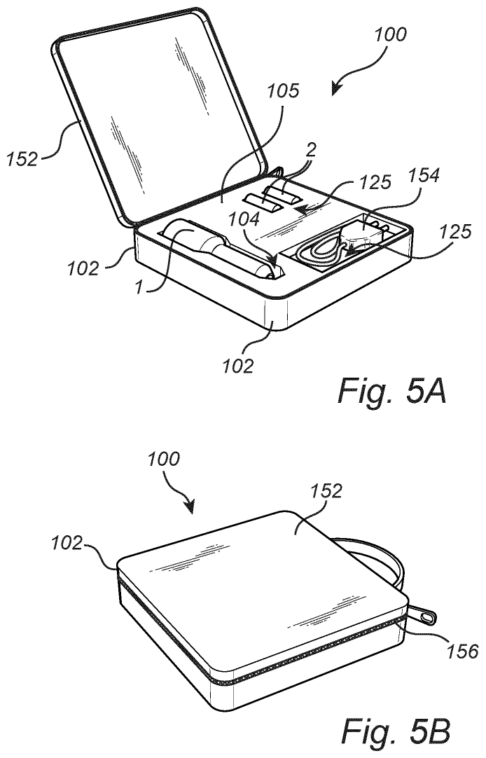

FIG. 5A and FIG. 5B illustrates a storage case 100 according to one embodiment. In FIG. 5A, the lid 152 is open and in FIG. 5B the lid 152 is closed.

The storage case 100 comprises an outer protective housing 102 to provide protection from shock, liquids, dust, dirt, and other contaminants that may cause inoperability or unintended operation of the medicament delivery device 1 stored inside the housing in a device storage portion 104. In the storage portions 125, medicament containers 2 are stored as well as a charger 154. The storage portions 125 and 104 may be formed in an inner tray 105. The storage case 100 may be sealed by a zipper 156.

The electronic components such as the controller circuitry, wiring, communication devices etc, described herein may be mounted under the tray 105.

A charging port (or opening for charging wire) may optionally provided on or through the outer surface of the outer protective housing 102 and provides a user with a connection point to charge the storage case internal battery and the medicament delivery device 1 contained within. Connectors such as mains power or preferably USB connections may be provided, optionally with a cover to prevent damage to the charging connector 112.

In certain use cases or environments, the outer housing and the openings and mating surfaces may contact liquids, dust, or dirt that may cause damage to or undesired operation of the circuits, electronic components, or a drug delivery device contained within. The outer protective housing 102 when assembled (or closed by a user of the apparatus) may be sealed to substantially prevent ingress of undesired materials. In some embodiments, the outer housing (when closed and/or latched) is designed to prevent fluid ingress according to a specific standard such as e.g., Ingress Protection Code defined in IEC 60529, or rating within such a standard such as e.g., IP54.

One or more components of the storage case such as e.g., controller 130, printed circuit boards, circuits 108, 118, battery 106, or other electronic components may be sealed against fluid intrusion by means of a conformal coating applied to one or more components of the storage case prior to or during assembly into the storage case. Conformal coatings may also be applied to one or more components of the apparatus to protect against unauthorized access to, disassembly of, or tampering with electronic circuitry and components within the apparatus.

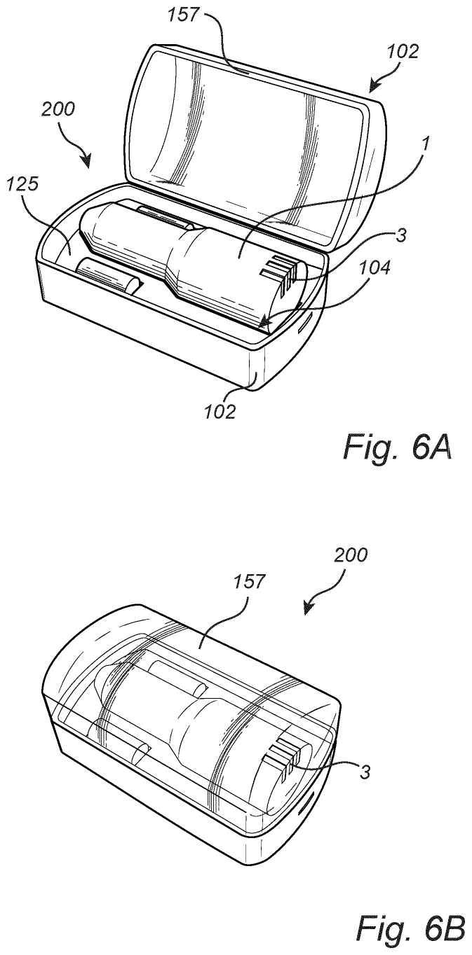

FIG. 6A and FIG. 6B illustrate an embodiment of the present disclosure. As above, the storage case 200 comprises a device storage portion 104 for accommodating a medicament delivery device 1 and a storage 125 for storing medicament containers 2. There is further shown a protective cover 102 and a hinged lid 157.

A part of the protective housing may be optically transmissive, or comprise a light transmissive window, and in the embodiment of FIGS. 6A-B, the lid 157 is optically transmissive. Through the optically transmissive lid 157, the at least one indicator on the medicament delivery device is visible from outside the protective housing 102. In other words, charging feedback may be provided through an indicator 3 or set of indicators 3 located on the medicament delivery device 1, such as blinking lights, numeric charging percentage, symbols, color coded LED segments, OLED display, or the like. Such indicators may be visible through a clear window, top lid, or other translucent or transparent feature, which may be optionally provided with indicia to help interpret the feedback, such as pad printed or silk-screened text or labelling.

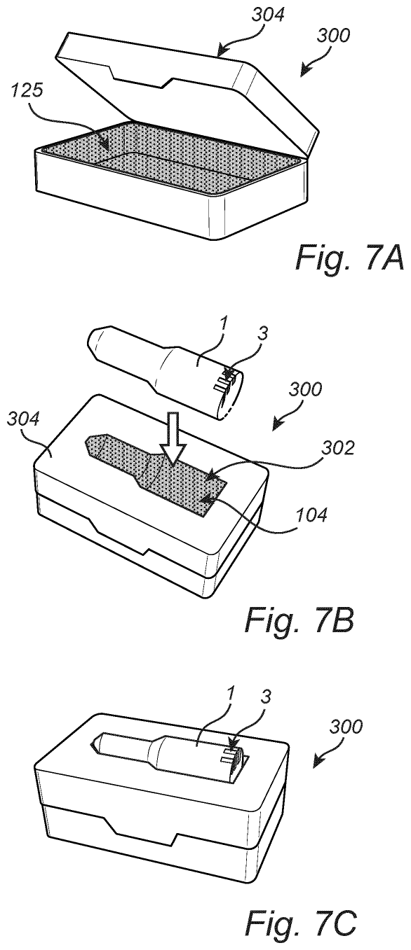

In an alternative embodiment shown in FIG. 7A-C, a cradle 302 is formed in the lid 304 as a device storage portion 104 as shown in FIG. 7B. The storage portion 125 shown in FIG. 7A is inside the storage case 300 as discussed with reference to FIGS. 6A-B. In the embodiment of FIG. 7A-C, the lid or top case 304 is not translucent. The indicators 3 may be used to communicate an injection state of the device during use by a patient, and to indicate charging state when the device 1 is being charged.

In the embodiments shown in FIGS. 6A-B and FIG. 7A-C, the medicament delivery device 1 may have two states: in case 200, 300 and out of case 200, 300. One set of indicators 3 may be used to monitor charging while in the case 200, 300 and provide user feedback regarding the injection when out of the case 200, 300. This simplifies complexity of the storage case and improves user intuitiveness, as the feedback cannot be misinterpreted.

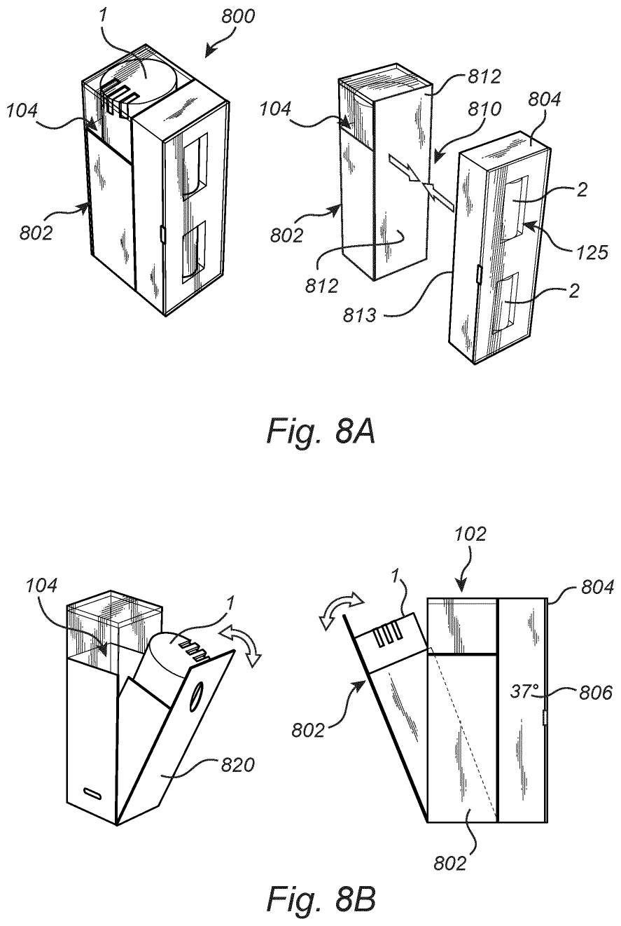

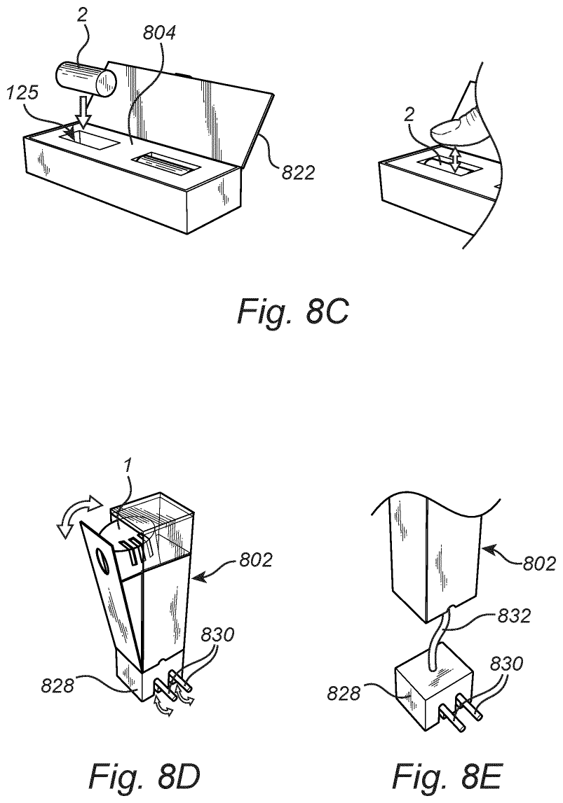

FIG. 8A-C illustrates an embodiment where a first portion 802 comprising the device storage portion 104 is separable from a second portion 804 comprising the medicament storage portion 125. The medicament delivery device 1 is chargeable by the internal battery of the first portion 802 as described above or using external charging input via a charging port of the first portion 802. The first portion 802 may include the charging circuitry 108, the internal battery 106, the communication arrangement 118, GPS 129, controller circuitry 130, charging port 112, and other electronic circuitry and components discussed above.

The second portion 804 comprises the medicament storage 125 for storing of medicament containers 2. The second portion 804 may optionally contain a smaller battery and separate circuitry and indicator 806 providing visual output of the time for injection, days to injection, temperature of the medication, or other parameters. The smaller battery may be inductively coupled to the device storage portion 104 to provide recharging.

In FIG. 8A, the first portion 802 and the second portion 804 are shown separated and joined. The portions 802 and 804 of the storage case 800 are reversibly separable from each other by a connection mechanism 810. For example, a magnetic connection may provide the connection mechanism. A magnetic element 812 or sheet on the first portion 802 may be attracted by a magnetic element 813 or sheet on the second portion 804 for connecting the two portions to each other.

Turning specially to FIG. 8B, the medicament delivery device 1 may be stored in a pocket of the first portion 802 that is configured as a push and release function. Thus, pushing on the lid 820 when it is in a closed state causes it is release and spring open.

Turning specially to FIG. 8C, the second portion comprises a lid 822 that can close the medicament storage portion 125. The medicament storage portion 125 is also provided with push-release functions to extract a medicament container from the medicament storage portion 125. The medicament storage portion 125 may be insulated to better preserve the medicament in the medicament storage containers 2.

As illustrated in FIG. 8D, optionally, the first portion 802 is equipped with an integral power plug 828 for directly connecting to the electrical mains for charging of the medicament delivery device 1. The plug 828 comprises electrically conductive elements 830 that may be hinged connected so that they may be pivoted into a storage position when not used.

The power plug may be releasable attached to the first portion 802 as illustrated in FIG. 8E. In this case, a retractable cord 832 electrically connects the elements with the first portion 802.

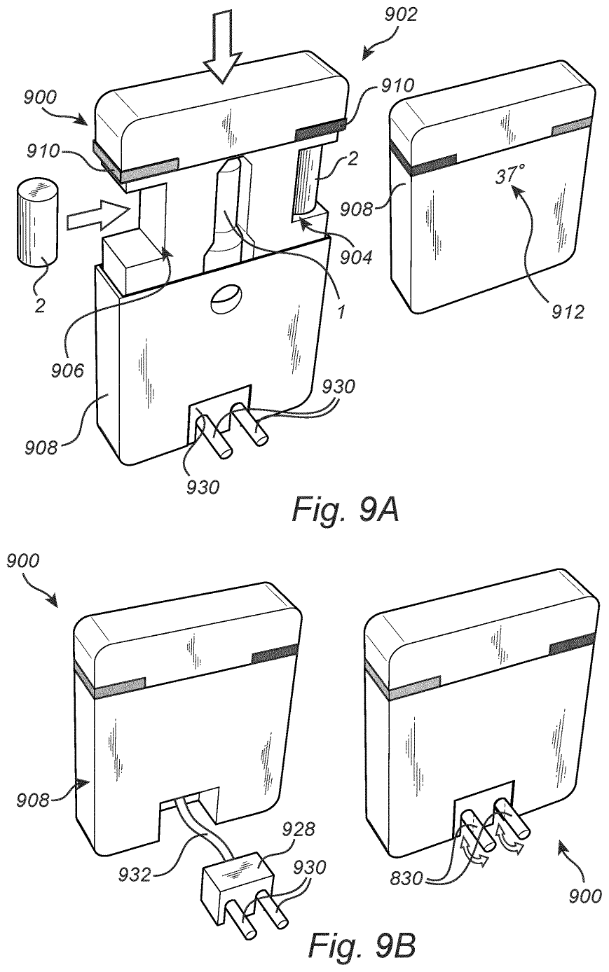

FIG. 9A illustrates another embodiment of the present disclosure. In this embodiment, the storage case 900 comprises a storage unit 902 that comprises the device storage portion 904 and a drug storage portion 906, the storage unit is slidable into the protective housing 908. The device storage portion 904 is located in a centre of the storage unit 902 and a drug storage portions 906 are located next to the device storage portion 904. The protective housing 908 surrounds the storage unit 902 when it is in a closed state. An indicator 912 of the device 900 provides visual output of the time for injection, days to injection, temperature of the medication, or other parameters. When in the closed state, indicators 910 are visible on the case 900. The storage unit 904 holding the medicament delivery device 1 is configured with internal light pipes that transmit light from the stored device 1 into the light pipes towards the unpowered indicators 910.

Turning to FIG. 9B the storage case 900 is equipped with a power plug 928 for directly connecting to the electrical mains for charging of the medicament delivery device 1. The plug 928 comprises electrically conductive elements 930 that may be hinged connected so that they may be pivoted into a storage position when not used.

The power plug 928 may be releasable attached to storage case 900 protective housing 908 as illustrated in FIG. 9B. A retractable cord 932 electrically connects the elements 930 with the circuitry inside the protective housing 908.

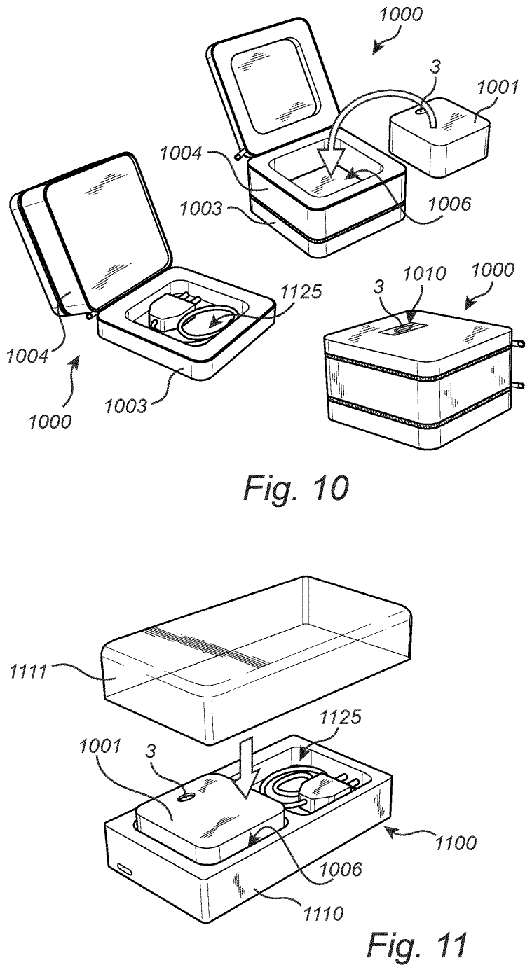

FIG. 10 illustrates a storage case 1000 for a medicament delivery device 1001 of a different form factor (e.g., the reusable component of a large volume injection device) and the device 1. The storage case 1000 comprises two zippers 1003, 1104, where zipper 1003 opens/seals a storage portion 1125 for e.g. medicament containers or chargers, etc. and upper zipper 1004 for opening/sealing a device storage portion 1006. The storage case 1000 comprises the charging circuitry 108, the internal battery 106, optionally the communication arrangement 118, optionally GPS 129, controller circuitry 130, charging port 112, and other electronic circuitry and components discussed above. In addition, a translucent window 1010 enables a user to see the indicators 3 used to display battery state during storage in the case 1000 as described above.

FIG. 11 illustrates a further possible storage case 1100 where the device storage portion 1006 and the storage portion 1125 are formed in the same tray 1110. A translucent or transparent lid 1111 allows for a suer to see the indicator when the lid 111 is closed.

If the device 1001 is provided with a screen, the screen may be similarly configured to display charging information through the storage case 1000 when stored, and injection related information when outside the case. Such a screen allows a more detailed amount of information to be provided to a user, such as time to full charge, visualization of battery level with graphics, indication of where charging is taking place from (case or mains power), and the like.

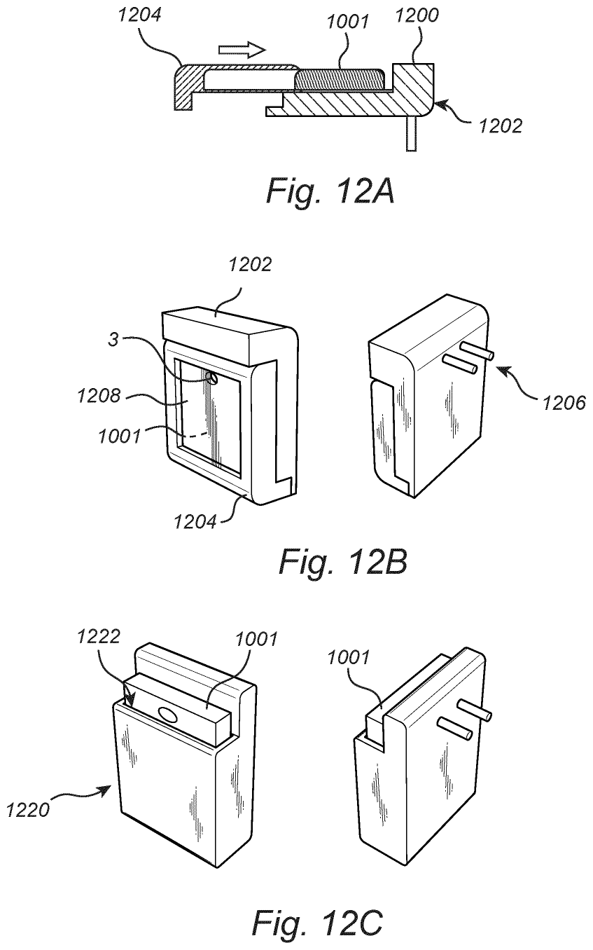

FIG. 12A-B illustrates a compact storage case 1200 comprising a device storage portion 1202 and a closable lid 1204. Best seen in the cross-section in FIG. 12A, the lid 1204 is slidable on the device storage portion 1202 to enclose the medicament delivery device 1001 via e.g., guiding rails on the device storage portion 1202 that matches with mating rails of the lid 1204.

The device storage portion further comprises an integral power plug 1206 for connection to the mains. Furthermore, the lid 1204 comprises a transparent window 1208 through which indicators 3 of the medicament delivery device 1001 are visible. The storage case 1200 further comprises the charging circuitry 108, the internal battery 106, optionally the communication arrangement 118, optionally a GPS 129, controller circuitry 130, charging port 112, and other electronic circuitry and components discussed above.

FIG. 12C illustrates a similar embodiment to that of FIG. 12A-B. However, in this embodiment the storage case 1220 includes a pocket 1222 in which the medicament delivery device 1001 is slid into place. An indicator 1226 of the storage case 1220 arranged along the rim of the opening of the pocket 1222 is connected to indicators of the device 1001 via light guides or pipes as described above so that the light indications of the device 1001 are guided to the visible indicator 1226 of the storage case 1220.

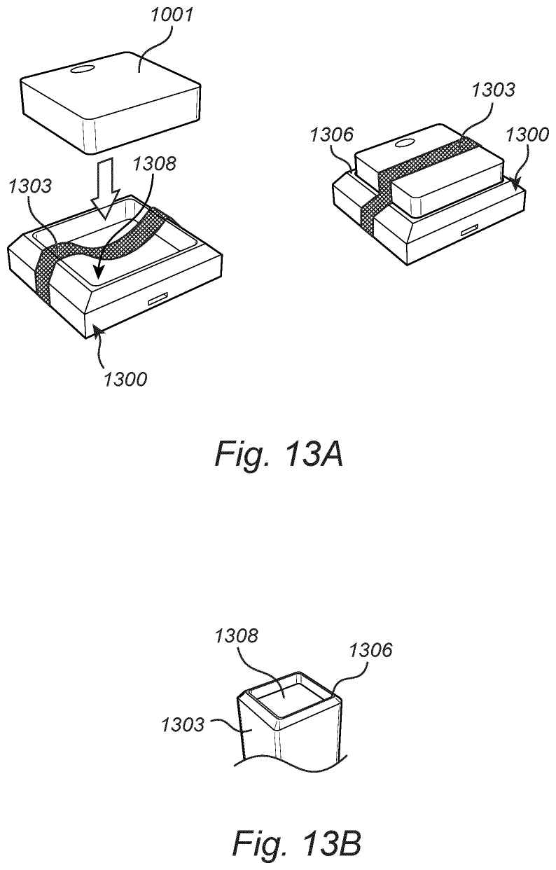

FIG. 13A-B illustrates a is a further possible implementation. In this storage case 1300, the medicament delivery device 1001 is strapped to the case 1300 using a strap 1303. Furthermore, the storage case 1300 includes a light indicator 1306 at the rim of the opening of the device storage portion 1308 that may be connected to indicators of the device 1001 via light guides or pipes as described above so that the light indications of the device 1001 are guided to the visible indicator 1306 of the storage case 1300.

The storage case 1300 further comprises the charging circuitry 108, the internal battery 106, optionally the communication arrangement 118, optionally a GPS 129, controller circuitry 130, charging port 112, and other electronic circuitry and components discussed above.

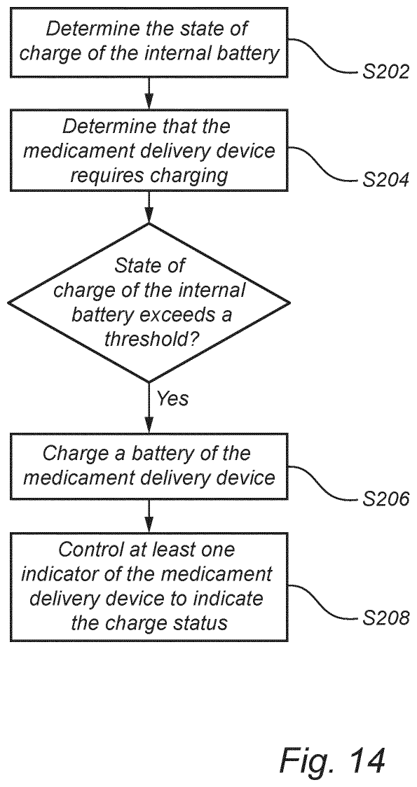

FIG. 14 is a flow chart of method steps according to one embodiment. The method is for controlling charging of an electrically rechargeable medicament delivery device storable in a storage case comprising: a device storage portion for accommodating the electrically rechargeable electromechanical medicament delivery device, and an internal battery and charging circuitry for charging of the medicament delivery device when housed in the device storage portion.

The method comprises, in step S202 determining the state of charge of the internal battery.

In step S204, determining that the medicament delivery device requires charging.

In case the state of charge of the internal battery exceeds a threshold, charging a battery of the medicament delivery device in step S206.

In step S208, controlling at least one indicator of the medicament delivery device to indicate the charge status of the medicament delivery device, the indicator being further configured to indicate injection status when the medicament delivery device is out of the storage case.

Charging the battery of the medicament delivery device is preferably performed only when the medicament delivery device is not simultaneously used for an injection event.

In some embodiments, the controller circuitry 130 receives a request for a present location of the storage case from a secondary electronic device via the communication circuitry 118. The present location is determined using the global positioning system 129 of the storage case. The controller circuitry 125 transmits, using the communication arrangement 118, from the storage case, a digital message to the secondary electronic device including information indicating the present location.

The storage case 100 may optionally comprise a solar panel 150 configured to covert light to electrical energy to electrically charge the internal battery or the medicament delivery device. This is useful in the absence of an electrical power source.

The storage case may further comprise an ultra-violet sterilizing device exposing the medicament delivery device to ultra-violet in the device storage. Ultra-violet LED sterilizing technology may thus be included to maintain the drug delivery device 1 free from any viruses, bacteria, fungi, and allergens that the device may be exposed to, especially during travel. Such technology may be manually activated by the user or automatically triggered when the drive unit is placed inside the charging storage case and the lid has been closed. An automatic shut-off feature may also be incorporated when a lid of the case is lifted to avoid unwanted UV light exposure (i.e., beyond a desired or controlled duration).

In addition to providing feedback on the charging status of the charging storage case internal battery and the reusable medicament delivery device battery, the storage case may also display additional feedback to the user. Such feedback may include, by way of example but not limitation, at least one of: available injection time based on charging percentage, remaining time to full charge, warm up time: triggered automatically by either the insertion or expulsion of the medication unit from the case, or manually initiated by the user, number of medication units inserted, medication status (i.e., discoloration, cloudiness, and particle detection, authenticity, expiry), medication frequency (i.e., days remaining until next dose), medication data (i.e., viscosity, and volume), medication temperature: Temperature monitoring may be provided through one or more low-cost, electronic temperature sensors printed into the charging storage case during manufacturing, device and medication authenticity, damage to the device such as dirt, water, or dust, electronic errors or malfunctions (device or charger). The supplementary indicators are independent from other features in the disclosure herein and any of the embodiments may encompass one, all or a combination of any of these feedback features. The feedback can be displayed, for example, through light patterns, haptic technology, sound, symbols, LED text, or display screens.

There is further provided processing circuitry 130 configured to control charging of an electrically rechargeable medicament delivery device storable in a storage case comprising a device storage portion for accommodating the electrically rechargeable electromechanical medicament delivery device, and an internal battery and charging circuitry for charging of the medicament delivery device when housed in the device storage portion, the processing circuitry is configured to: determine the state of charge of the internal battery; determine that the medicament delivery device requires charging, in case the state of charge of the internal battery exceeds a threshold, charging a battery of the medicament delivery device, and control at least one indicator of the medicament delivery device to indicate the charge status of the medicament delivery device, the indicator being further configured to indicate injection status when the medicament delivery device is out of the storage case.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the support pad are configured to recognize the event and give feedback to the patient via haptic, visual, or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, as it can be used as a plug-and-play device and no prior setup is needed; however, it may be more expensive or require a larger PCB.

Depending on the requirements of the product any of those two technologies can be used.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are defined by the following clauses.
1. A storage case for an electromechanical medicament delivery device, the storage case comprising:
   an outer protective housing;
   a device storage portion for an electrically rechargeable electromechanical medicament delivery device,
   an internal battery and a charging circuitry for charging of the internal battery of the storage case and a battery of the medicament delivery device when housed in the device storage portion,
   a charging input device connected with the charging circuitry of the internal battery and with a charging interface in the device storage portion, and
   controller circuitry configured to:
   determine the state of charge of the internal battery;
   determine that the medicament delivery device requires charging,
   in case the state of charge of the internal battery exceeds a threshold, control the charging circuitry to charge a battery of the medicament delivery device,
   control at least one indicator of the medicament delivery device to indicate the charge status of the medicament delivery device, the indicator being configured to indicate injection status when the medicament delivery device is out of the storage case.
2. The storage case according to clause 1, the controller circuitry being further configured to:
   detect that no medicament delivery device is in the device storage portion;
   determine that the state of charge of the internal battery is below a recharging limit,
   control the charging circuit to recharge the internal battery with power provided at the charging input device, and
   control at least one of the indicators to indicate the charge status of the internal battery only if the medicament delivery device is not simultaneously used for an injection event.
3. The storage case according to any one of clauses 1 and 2,
   control the charging circuit to recharge the battery of the medicament delivery device only when the medicament delivery device is not simultaneously used for an injection event.
4. The storage case according to any one of the preceding clauses, the controller circuitry being further configured to:
   if the medicament delivery device is in the device storage portion, and the charging input device receives electrical power from an external power source, control a switching circuitry to bypass the internal battery and provide charging power directly to the medicament delivery device.
5. The storage case according to any one of the preceding clauses, the controller circuitry being further configured to:
   determine that an injection event is due within a predetermined time, and
   control the charging circuitry to charge the battery of the medicament delivery device to at least a minimum state of charge required for the next injection event.
6. The storage case according to any one of the preceding clauses, comprising a global positioning system.
7. The storage case according to any one of the preceding clauses, comprising a wireless communication device configured to receive and transmit data to a remote server, the controller circuitry being further configured to:
   collect injection data from the medicament delivery device, and
   transmit the injection data using the wireless communication device to the remote server.
8. The storage case according to clause 7, the controller circuitry being further configured to:
   receive firmware updates for the medicament delivery device using the wireless communication device,
   optionally, compare the current installed firmware upon the medicament delivery device to the received firmware update,
   update the firmware of the medicament delivery device.
9. The storage case according to clause 8, the controller circuitry being further configured to:
   compare the current installed firmware upon the medicament delivery device to the received firmware update.
10. The storage case according to any one of the preceding clauses, comprising a drug storage portion for storage of a medicament container inside the protective housing
11. The storage case according to clause 10, comprising a refrigerator device to refrigerate the drug storage portion.
12. The storage case according to any one of clauses 10 and 11, wherein the storage case comprises two portions reversibly separable from each other by a connection mechanism, wherein a first portion comprises the device storage portion and a second portion comprises a drug storage portion.
13. The storage case according to clause 12, the first portion further comprising a power plug for directly connecting to the electrical mains for charging of the medicament delivery device.
14. The storage case according to clause 12, comprising a retractable power cord between the power plug and the first portion, wherein the power plug is releasable attached to the first portion.
15. The storage case according to any one of the preceding clauses, wherein the protective housing comprises an at least partly light transmissive window through which the at least one indicator on the medicament delivery device is visible from outside the protective housing.
16. The storage case according to any one of the preceding clauses, comprising a storage unit comprising the device storage portion and a drug storage portion, the storage unit is slidable into the protective housing.
17. The storage case according to any one of the preceding clauses, comprising a solar panel configured to covert light to electrical energy to electrically charge the internal battery or the medicament delivery device.
18. The storage case according to any one of the preceding clauses, comprising an ultra-violet sterilizing device exposing the medicament delivery device to ultra-violet in the device storage.
19. The storage case according to any one of the preceding clauses, comprising:
   at least one storage compartment for drug cassettes and RFID antennas, the RFID antennas are configured to communicate with RFID chips on the drug cassettes to communicate information to the medicament delivery device.
20. A method for controlling charging of an electrically rechargeable medicament delivery device storable in a storage case comprising:
   a device storage portion for accommodating the electrically rechargeable electromechanical medicament delivery device, and
   an internal battery and charging circuitry for charging of the medicament delivery device when housed in the device storage portion, the method comprising:
   determining the state of charge of the internal battery;
   determining that the medicament delivery device requires charging,
   in case the state of charge of the internal battery exceeds a threshold, charging a battery of the medicament delivery device, and
   controlling at least one indicator of the medicament delivery device to indicate the charge status of the medicament delivery device, the indicator being further configured to indicate injection status when the medicament delivery device is out of the storage case.
21. The method according to clause 20, further comprising:
   detecting that no medicament delivery device is in the device storage portion;
   determining that the state of charge of the internal battery is below a recharging limit,
   charging the internal battery with power provided at a charging input device connected to external mains power, and
   controlling at least one of the indicators to indicate the charge status only if the medicament delivery device is not simultaneously used for an injection event.
22. The method according to any one of clauses 20 and 21, comprising:
   if the medicament delivery device is in the device storage portion, and if electrical power is being received from an external power source, controlling a switching circuitry to bypass the internal battery and provide charging power directly to the medicament delivery device.
23. The method according to any one of clauses 20 to 22, further comprising:
   determining that an injection event is due within a predetermined time, and
   charging the battery of the medicament delivery device to at least a minimum state of charge required for the next injection event.
24. The method according to any one of clauses 20 to 23, further comprising:
   receiving a request for a present location of the storage case from a secondary electronic device;
   determining the present location using a global positioning system of the storage case, and
   transmitting, from the storage case, a digital message to the secondary electronic device including information indicating the present location.
25. The method according to any one of clauses 20 to 24, comprising:
   charging the battery of the medicament delivery device only when the medicament delivery device is not simultaneously used for an injection event.
26. Processing circuitry configured to control charging of an electrically rechargeable medicament delivery device storable in a storage case comprising:
   a device storage portion for accommodating the electrically rechargeable electromechanical medicament delivery device, and
   an internal battery and charging circuitry for charging of the medicament delivery device when housed in the device storage portion, the processing circuitry is configured to:
   determine the state of charge of the internal battery;
   determine that the medicament delivery device requires charging,
   in case the state of charge of the internal battery exceeds a threshold, charging a battery of the medicament delivery device, and
   control at least one indicator of the medicament delivery device to indicate the charge status of the medicament delivery device, the indicator being further configured to indicate injection status when the medicament delivery device is out of the storage case.

1-15. (canceled)

16. A storage case for an electromechanical medicament delivery device, the storage case comprising:
an outer protective housing;
a device storage portion for an electrically rechargeable electromechanical medicament delivery device;
an internal battery and a charging circuitry for charging of the internal battery of the storage case and a battery of the medicament delivery device when housed in the device storage portion;
a charging input device connected with the charging circuitry of the internal battery and with a charging interface in the device storage portion; and
controller circuitry configured to:
determine the state of charge of the internal battery;
determine that the medicament delivery device requires charging;
in case the state of charge of the internal battery exceeds a threshold, control the charging circuitry to charge a battery of the medicament delivery device; and
control at least one indicator of the medicament delivery device to indicate the charge status of the medicament delivery device, the indicator being configured to indicate injection status when the medicament delivery device is out of the storage case.

17. The storage case according to claim 16, wherein the controller circuitry is further configured to:
detect that no medicament delivery device is in the device storage portion;
determine that the state of charge of the internal battery is below a recharging limit;
control the charging circuit to recharge the internal battery with power provided at the charging input device; and
control at least one of the indicators to indicate the charge status of the internal battery only if the medicament delivery device is not simultaneously used for an injection event.

18. The storage case according to claim 16, wherein the controller circuitry is further configured to:
control the charging circuit to recharge the battery of the medicament delivery device only when the medicament delivery device is not simultaneously used for an injection event.

19. The storage case according to claim 16, wherein the controller circuitry is further configured to:
if the medicament delivery device is in the device storage portion, and the charging input device receives electrical power from an external power source, control a switching circuitry to bypass the internal battery and provide charging power directly to the medicament delivery device.

20. The storage case according to claim 16, wherein the controller circuitry is further configured to:
determine that an injection event is due within a predetermined time; and
control the charging circuitry to charge the battery of the medicament delivery device to at least a minimum state of charge required for the next injection event.

21. The storage case according to claim 16, further comprising a wireless communication device configured to receive and transmit data to a remote server, wherein the controller circuitry is further configured to:
collect injection data from the medicament delivery device; and
transmit the injection data using the wireless communication device to the remote server.

22. The storage case according to claim 21, wherein the controller circuitry is further configured to:
receive firmware updates for the medicament delivery device using the wireless communication device;
optionally, compare the current installed firmware upon the medicament delivery device to the received firmware update; and
update the firmware of the medicament delivery device.

23. The storage case according to claim 22, wherein the controller circuitry is further configured to:
compare the current installed firmware upon the medicament delivery device to the received firmware update.

24. The storage case according to claim 16, further comprising a drug storage portion for storage of a medicament container inside the protective housing.

25. The storage case according to claim 24, further comprising a refrigerator device to refrigerate the drug storage portion.

26. The storage case according to claim 16, further comprising:
at least one storage compartment for drug cassettes and RFID antennas, the RFID antennas are configured to communicate with RFID chips on the drug cassettes to communicate information to the medicament delivery device.

27. A method for controlling charging of an electrically rechargeable medicament delivery device storable in a storage case comprising a device storage portion for accommodating the electrically rechargeable electromechanical medicament delivery device, and an internal battery and charging circuitry for charging of the medicament delivery device when housed in the device storage portion, the method comprising:
determining the state of charge of the internal battery;
determining that the medicament delivery device requires charging;
in case the state of charge of the internal battery exceeds a threshold, charging a battery of the medicament delivery device; and
controlling at least one indicator of the medicament delivery device to indicate the charge status of the medicament delivery device, the indicator being further configured to indicate injection status when the medicament delivery device is out of the storage case.

28. The method according to claim 27, further comprising:
detecting that no medicament delivery device is in the device storage portion;
determining that the state of charge of the internal battery is below a recharging limit;
charging the internal battery with power provided at a charging input device connected to external mains power; and
controlling at least one of the indicators to indicate the charge status only if the medicament delivery device is not simultaneously used for an injection event.

29. The method according to claim 27, further comprising:
if the medicament delivery device is in the device storage portion, and if electrical power is being received from an external power source, controlling a switching circuitry to bypass the internal battery and provide charging power directly to the medicament delivery device.

30. The method according to claim 27, further comprising:
determining that an injection event is due within a predetermined time, and
charging the battery of the medicament delivery device to at least a minimum state of charge required for the next injection event.
